(19) United States
(12) Patent Application Publication
(10) Pub. No.: US 2009/0054923 A1
(43) Pub. Date: Feb. 26, 2009

(54) Nasal Device

(75) Inventor: Richard Benson, Queensland (AU)

Correspondence Address: SHOEMAKER AND MATTARE, LTD, 10 POST OFFICE ROAD - SUITE 100, SILVER SPRING, MD, 20910, US

(73) Assignee: BENSON MEDICAL SERVICES PTY LTD, Capalaba, Brisbane, QLD (AU)

(21) Appl. No.: 11/718,355

(22) PCT Filed: Aug. 16, 2005
(86) PCT No.: PCT/AU2005/001221

§ 371 Date: May 1, 2007

(30) Foreign Application Priority Data
Nov. 1, 2004 (AU) 2004100927

(51) Int. Cl. A61M 29/02

(52) U.S. Cl. 606/199

(57) ABSTRACT

The current invention is directed to a nasal device for maintaining an air passageway within the nose while a person sleeps. The nasal device has two spaced apart tubular members joined together by a bridging section and a removable spring biased clip that is locatable to hold it in position. The clip of the nasal device prevents the device from being accidentally dislodged and can also be used by people with different nose and nasal septum size and shape.

### FIELD OF INVENTION

The present invention relates to a device for assisting breathing through the nose particularly for use while a person sleeps. The present invention has particular but not exclusive application for treating snoring and sleep apnea conditions.

### BACKGROUND OF THE INVENTION

There are a number of nasal devices that have been developed to assist a person with their breathing while they are asleep. These nasal devices usually dilate or maintain the air passageway within each nasal cavity. In U.S. Pat. No. 5,895,489 there is described a nasal dilator having two open framework tubes connected together with a bridge section. The frames are insertable into the nostrils but deep nasal insertion is prevented by a larger external end contacting the narrowing nasal passage and preventing further insertion. U.S. Pat. No. 6,562,057 discloses another similar nasal device having tubular elements with elongate apertures and a curved bridge section that maintains the tubular elements in a spaced apart and substantially parallel relationship. German patent specification DE29616121 discloses a nasal device having two tubular members and an alternate bridge section. Another similar nasal device was disclosed in Swiss patent specification CH689199. The nasal device consisted of two tubes 10 mm to 13 mm long and molded in one piece with a connecting filament from silicon.

The disclosed nasal devices however can become accidentally dislodged and fall out or repositioned with reduced effectiveness. Furthermore where the nose or nasal septum have a different size or shape or a person has a deviated septum, current nasal devices may not fit or comparatively be more easily dislodged.

### OBJECT OF THE INVENTION

It is an object of the present invention to provide an alternate nasal device for maintaining an air passageway within the nose that overcomes at least in part one or more of the abovementioned problems.

### SUMMARY OF THE INVENTION

In one aspect the present invention broadly resides in a nasal device for maintaining an air passageway within the nose including

a dilator member having two nasal members integral with or connectable to a bridging member, said bridging member joins the two nasal members so that they are in similar orientation and substantially parallel with each other, each of said nasal members are shaped to form an air passageway when inserted within a nasal cavity; and

a spring biased clip positionable on each of the nasal members wherein in use the two nasal members within respective nostril cavities form the air passageways and are maintained adjacent the nasal septum by the spring biased clip.

The nasal member may be any suitable shape which in use is able to form an air passageway within the nasal cavity. Preferably each of the nasal members is hollow and substantially tubular in shape. Each of the nasal members preferably has an internal bore extending the length of the member. Preferably each of the nasal members has a truncated conical shape where the diameter across one end is smaller than the diameter across the opposite end. Preferably each of the nasal members has a truncated conical shape wherein there is a bore extending the length of the member. The narrower end of the nasal member is preferably positioned further within the nasal cavity than the larger end when the dilator member is inserted within the nose. Preferably the narrower end of the nasal member has a rounded edge to reduce the risk of damage to nasal tissue.

Each nasal member preferably has a locating recess on an internal wall for the positioning of the clip.

Preferably the bridging member is connectable to or adjacent the larger end of each nasal member.

The bridging member is preferably integral with the nasal members to form the dilator member. Alternately the bridging member includes a separable bridge portion that can attach onto complementary protrusions on each of the nasal members.

The bridging member preferably includes a tab extending outwardly from the nasal member. The tab preferably enables a person to hold and withdraw the nasal device from the nose.

The dilator member preferably is made of soft material. The dilator member is preferably made of soft, resiliently deformable material. The dilator member preferably is made of plastics. The dilator member is preferably made of non-toxic, non-abrasive, soft medical plastics to minimize the risk of damage to nasal tissue.

The spring biased clip is preferably substantially U-shaped. The spring biased clip preferably has locating protrusions on opposing clip ends. The locating protrusions preferably oppose each other. The locating protrusions assist in positioning the clip by locating within the respective locating recess in each nasal member. When the spring biased clip is located within each nasal member, the clip preferably abuts each nasal member substantially against the nasal septum wall. When in position the clip preferably prevents accidental dislodgement of the nasal members in the nasal cavities and movement of the nasal members by airflow with inhalation and expiration.

The spring biased clip is preferably made of hard and stiff material. The spring biased clip is preferably made of surgical steel.

In another aspect the present invention broadly resides in a nasal device for maintaining an air passageway within the nose including

a dilator member having two nasal members integral with a bridging member, said bridging member positions the two nasal members so that they are in similar orientation and substantially parallel with each other, each of said nasal members are hollow and substantially tubular in shape; and

a spring biased clip positionable on each of the nasal members, wherein in use the two nasal members within respective nostril cavities form air passageways and are maintained adjacent the nasal septum by the spring biased clip.

Preferably each of the nasal members has a substantially truncated conical shape where the diameter across one end is smaller than the diameter across the opposite end. Preferably each of the nasal members has a substantially truncated conical shape wherein there is a bore extending the length of the member. Preferably the narrower end of the nasal member has a rounded edge to reduce the risk of damage to nasal tissue.

Other features described with reference to the first aspect may also be applicable to the second aspect of the nasal device.

### BRIEF DESCRIPTION OF THE DRAWINGS

In order that the present invention can be more readily understood and put into practical effect, reference will now be made to the accompanying drawings wherein:

FIG. 1 is a perspective view of the dilator member of the preferred embodiment of the nasal device;

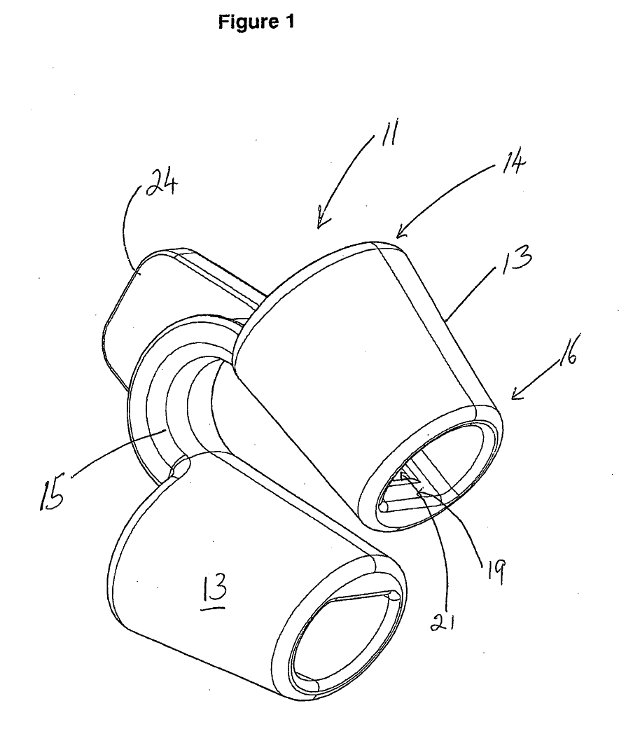

FIG. 2 is a diagrammatic view of the preferred embodiment of the nasal device;

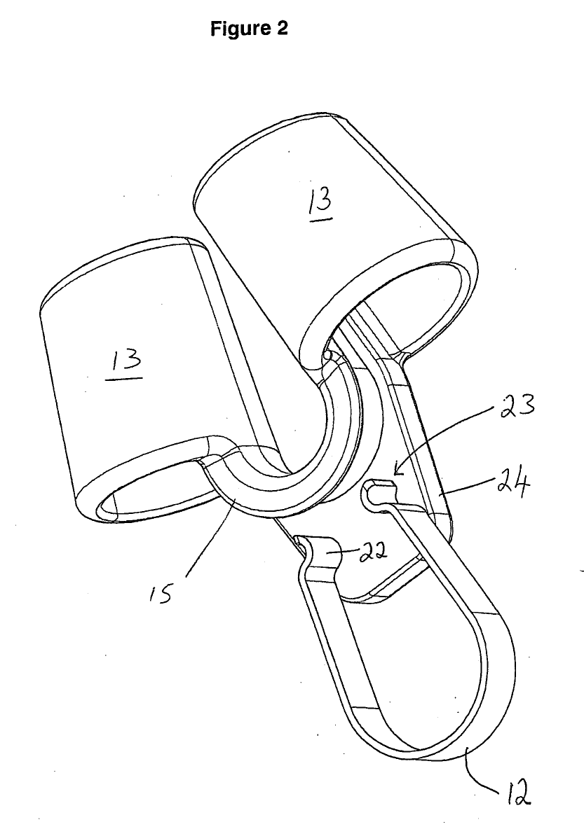

FIG. 3 is a plan diagrammatic view of the preferred embodiment of the nasal device;

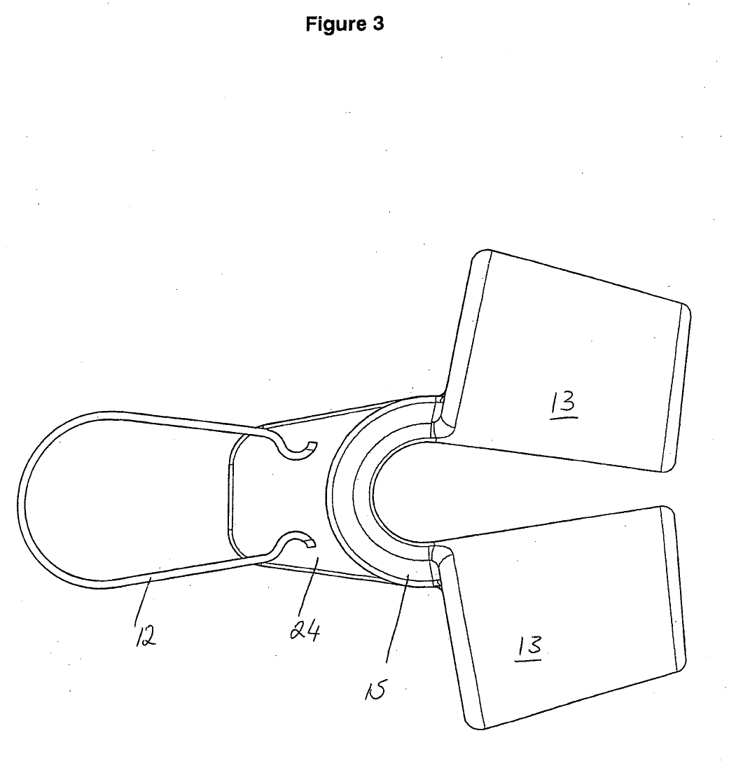

FIG. 4 is a plan diagrammatic view of the dilator member of the preferred embodiment of the nasal device; and

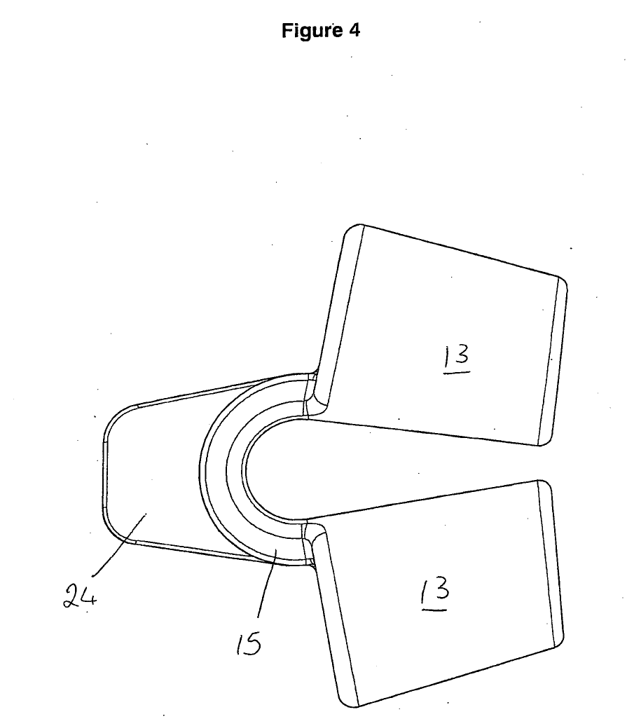

FIG. 5 is a sectional view of the preferred embodiment of the nasal device with the clip in the operational position.

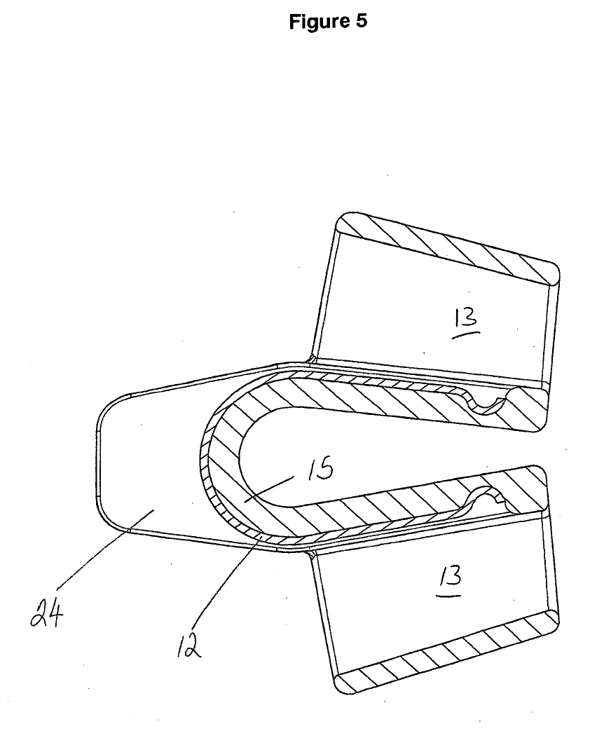

### DETAILED DESCRIPTION OF THE PREFERRED EMBODIMENT

With reference to the figures there is shown a nasal device 10 including a dilator member 11 and spring biased clip 12. The dilator member 11 has two hollow tubular nasal members 13. Each nasal member 13 decreases in diameter to form an elongate truncated cone. The larger end 14 of each nasal member 13 is joined by a bridging portion 15 which maintains the nasal members 13 in a similar orientation and in spaced relationship with each other. The bridging portion 15 has a tab 17 allowing a person to hold and withdraw the nasal device 10. The tab 17 extends outwardly from the nasal members 13. The internal wall 19 of each of the nasal members 13 has a locating recess 21 for the positioning and fixing of the spring biased clip 12.

The spring biased clip 12 is substantially U-shaped having opposing locating protrusions 22 on opposing ends 23. The clip 12 is made of surgical grade spring steel while the dilator member 11 is made of non-toxic non-abrasive medical grade plastics or latex-silicone.

The nasal device preferably has smooth surfaces and rounded edges to minimize risk of soft tissue damage and ulcer formation.

In use the nasal device 10 is used to maintain the nasal air passages during sleep. The dilator member 11 is inserted into the nose with the nasal members 13 placed within respective nasal cavities. The narrower end 16 of the nasal member 13 is inserted further in the nasal cavities than the larger end 14. The narrower end 16 has rounded edges 18 to minimize risk of tissue damage. The dilator member 11 is comfortably positioned and is prevented from extending too far into the nasal cavities by the bridging portion 15 which spans the nasal septum. The dilator member 13 is then secured in position by the spring biased clip 12 which is placed along the bridge portion 15, into the nasal members 13 and fixed by positioning the locating protrusions 22 on the clip 12 within the locating recesses 21 of the nasal members 13. The bridging portion 15 has an outwardly extending tab 24 which can be held and used to position the nasal device 10 or withdraw the nasal device 10 after use. After use the nasal device 10 is cleaned and stored for future use.

### ADVANTAGES

The preferred embodiment of the nasal device has two spaced apart tubular members joined together by a bridging section and a removable spring biased clip that is locatable to hold it in position. The preferred embodiment of the nasal device has the advantage that it is substantially prevented from being accidentally dislodged and can be used by people with different nose and nasal septum size and shape. Thus the nasal device has comparatively greater reliability in use and is suitable for use by a greater number of people. The preferred embodiment of the nasal device also provides the advantage that the clip prevents accidental dislodgement of the dilator member and thereby minimizes the risk of the dilator member falling from the nasal passages and into the mouth which may cause choking. The clip in this respect also serves as a safety device.

### VARIATIONS

It will of course be realised that while the foregoing has been given by way of illustrative example of this invention, all such and other modifications and variations thereto as would be apparent to persons skilled in the art are deemed to fall within the broad scope and ambit of this invention as is herein set forth.

Throughout the description and claims this specification the word “comprise” and variations of that word such as “comprises” and “comprising”, are not intended to exclude other additives, components, integers or steps.

1-17. (canceled)

18. A nasal device for maintaining an air passageway within the nose including
a dilator member having two nasal members integral with a bridging member, said bridging member positions the two nasal members so that they are in similar orientation and substantially parallel with each other, each of said nasal members are hollow and substantially tubular in shape; and
a spring biased clip positionable on each of the nasal members, wherein in use the two nasal members within respective nostril cavities form air passageways and are maintained adjacent the nasal septum by the spring biased clip.

19. A nasal device for maintaining an air passageway within the nose including
a dilator member having two nasal members integral with or connectable to a bridging member, said bridging member joins the two nasal members so that they are in similar orientation and substantially parallel with each other, each of said nasal members are shaped to form an air passageway when inserted within a nasal cavity; and
a spring biased clip positionable on each of the nasal members wherein in use the two nasal members within respective nostril cavities form the air passageways and are maintained adjacent the nasal septum by the spring biased clip.

20. A nasal device as claimed in claim 19 wherein each of the nasal members is hollow and substantially tubular in shape.

21. A nasal device as claimed in claim 19 wherein each of the nasal members is hollow and substantially tubular in shape and each of the nasal members has a truncated conical shape where the diameter across one end is smaller than the diameter across the opposite end.

22. A nasal device as claimed in claim 19 wherein each of the nasal members is hollow and substantially tubular in shape, each of the nasal members has a truncated conical shape where the diameter across one end is smaller than the diameter across the opposite end and the narrower end of the nasal member has a rounded edge to reduce the risk of damage to nasal tissue.

23. A nasal device as claimed in claim 19 wherein each of the nasal members is hollow and substantially tubular in shape, each nasal member has a locating recess on an internal wall for the positioning of the clip and said clip has complementary locating protrusions.

24. A nasal device as claimed in claim 19 wherein each of the nasal members is hollow and substantially tubular in shape and the bridging member is connectable to or adjacent the larger end of each nasal member.

25. A nasal device as claimed in claim 19 wherein each of the nasal members is hollow and substantially tubular in shape and the bridging member is integral with the nasal members to form the dilator member.

26. A nasal device as claimed in claim 19 wherein each of the nasal members is hollow and substantially tubular in shape, the bridging member includes a tab extending outwardly from the nasal member and said tab enables a person to hold and withdraw the nasal device from the nose.

27. A nasal device as claimed in claim 19 wherein each of the nasal members is hollow and substantially tubular in shape, the bridging member is integral with the nasal members to form the dilator member and the dilator member is made of non-toxic, non-abrasive, soft medical plastics to minimize the risk of damage to nasal tissue.

28. A nasal device as claimed in claim 19 wherein each of the nasal members is hollow and substantially tubular in shape and the spring biased clip is substantially U-shaped.

29. A nasal device as claimed in claim 19 wherein each of the nasal members is hollow and substantially tubular in shape and the spring biased clip has locating protrusions on opposing clip ends, said locating protrusions oppose each other.

30. A nasal device as claimed in claim 19 wherein each of the nasal members is hollow and substantially tubular in shape and the spring biased clip abuts each nasal member substantially against the nasal septum wall, when the clip is located within each nasal member.

31. A nasal device as claimed in claim 19 wherein each of the nasal members is hollow and substantially tubular in shape and the spring biased clip substantially prevents accidental dislodgement of the nasal members in the nasal cavities and movement of the nasal members by airflow with inhalation and expiration.

32. A nasal device as claimed in claim 19 wherein each of the nasal members is hollow and substantially tubular in shape and the spring biased clip is made of hard and stiff material.

33. A nasal device as claimed in claim 19 wherein each of the nasal members is hollow and substantially tubular in shape and the spring biased clip is made of surgical steel.
